(19) United States
(12) Patent Application Publication
(10) Pub. No.: US 2009/0187218 A1
(43) Pub. Date: Jul. 23, 2009

(54) Bone fixation plate with wire members for resisting back out of bone anchors

(75) Inventor: Cory R. Schaffhausen, Salt Lake City, UT (US)

Correspondence Address: Richard G. Gervase;Mintz Levin Cohn Ferris Glovsky and Popeo PC, Chrysler Center, 666 Third Avenue, 24th Floor, New York, NY, 10017, US

(73) Assignee: Amedica Corporation, Salt Lake City, UT (US)

(21) Appl. No.: 12/009,545

(22) Filed: Jan. 17, 2008

(51) Int. Cl. A61B 17/80; A61B 17/56

(52) U.S. Cl. 606/286; 606/280; 606/70

(57) ABSTRACT

A bone (e.g., spine) fixation plate with one or more wire members is provided. In some embodiments, each wire member spans across a top surface of the bone fixation plate in a direction generally transverse to a longitudinal axis of the plate. The wire member may flex (e.g., elastically) between a first position in which the wire member permits advancement of the bone anchor (e.g., screw) past the member, partially through the plate, and into bone, and a second position in which the wire member resists back out of the bone anchor from the plate.

### FIELD OF THE INVENTION

Embodiments of the present invention relate to bone fixation plates, and more particularly, to bone (e.g., spine) fixation plates with wire members that resist back out of associated bone anchors (e.g., screws).

### BACKGROUND OF THE INVENTION

The spine is a flexible, multi-segmented column that supports upright posture in a human while providing mobility to the axial skeleton. The spine encases and protects vital neural elements while providing structural support for the body by transmitting the weight of the body through the pelvis to the lower extremities. The cervical spine exhibits a wide range of motion due to the orientation of its facets and the lack of supporting structures. The thoracic and lumbar regions of the spine also have a significant range of motion.

The spine is made up primarily of bone and intervertebral discs, which are surrounded by supporting ligaments, muscle, fascia, blood vessels, nerves, and skin. These elements are subject to a variety of pathological disturbances: inflammation, trauma, neoplasm, congenital anomalies, disease, etc. Trauma to the spine can play a large role in the etiology of neck and low back pain. For example, trauma frequently results in damage at the upper end of the lumbar spine, where the mobile lumbar segments join the less mobile dorsal spine. Excessive forces on the spine not only produce life-threatening traumatic injuries, but may contribute to an increased rate of degenerative change.

The cervical region of the spine comprises the seven most superior vertebrae of the spine, which begin at the base of the skull and end at the upper torso. Because the neck has a wide range of motion and is the main support for the head, the neck is extremely vulnerable to injury and degeneration.

Spinal fixation is a common method of treating spinal disorders, fractures, and degeneration. One common device used for spinal fixation is the bone fixation plate, which is typically used in conjunction with a graft device placed between the vertebral bodies. Generally, there are two types of spinal plates: (i) constrained plates and (ii) semiconstrained plates. Generally, a constrained plate completely immobilizes the vertebrae and does not allow for graft settling. In this instance, the plate itself carries a significant portion of the loading. Constrained plates are useful, for example, in patients with highly unstable anatomy, such as with a vertebrectomy, or in patients with little chance of bone growth, such as cancer patients. In contrast, a semiconstrained plate is dynamic and allows for a limited degree of graft settling through micro-adjustments made between the plate and bone screws attaching the plate to the spine. The operation of the semiconstrained plate stimulates bone growth because the loading is transferred through the graft. Each type of plate has its own advantages depending upon the anatomy and age of the patient, and the results desired by the surgeon.

A typical bone fixation plate includes a relatively flat, rectangular plate having a plurality of apertures formed therein. A corresponding plurality of bone screws may be provided to secure the bone fixation plate to the vertebrae of the spine. A common problem associated with such a bone fixation plate is the tendency for bone screws to become dislodged from the bone and “back out” from the plate, thereby causing the plate to loosen and the screws to protrude from the plate. For example, in a typical anterior cervical fusion surgery, the carotid sheath, sternocleidomastoid muscles, trachea, and esophagus are moved laterally in order to expose the cervical spine. The cervical plate is designed to lie on the anterior face of the spine, dorsal to the esophagus. Due to its relative location to the esophagus and other connective tissue, if the bone screw securing the plate to the cervical spine backs out, the bone screw could pierce the esophagus, causing not only pain and infection, but also posing a serious risk of death to the patient. Bone fixation plates with large anterior-posterior profiles (e.g., thickness) can also make it difficult for the patient to swallow post-surgery.

In view of the foregoing, it would be desirable to provide bone fixation assemblies that resist back out of associated bone anchors. It would also be desirable to provide bone fixation assemblies that have reduced anterior-posterior profiles.

### SUMMARY OF THE INVENTION

Embodiments of the present invention relate to bone (e.g., spine) plating systems that resist back out of associated bone anchors.

In an aspect, a bone fixation assembly is provided that includes a bone fixation plate and at least one wire member for retaining one or more bone anchors (e.g., screw) within the plate. The bone fixation plate includes a top surface, a bottom surface, and at least one aperture (e.g., having a circular cross-section) between the top and bottom surfaces for permitting partial passage of a bone anchor through the plate. The bone fixation plate has a length in one direction and a width in another direction, where the width is less than the length. The wire member includes one or more elongate arms that span across the top surface of the plate in a direction substantially transverse to the lengthwise direction of the plate. For example, the elongate arm(s) may extend across the entire width of the plate. The one or more elongate arms at least partially cover a proximal end (e.g., head) of the bone anchor when the bone anchor is positioned within the plate.

In some embodiments, the one or more elongate arms may be configured to flex (e.g., at least partially elastically) between (i) a first position in which a bone anchor can be advanced past the one or more elongate arms, partially through the plate, and into bone, and (ii) a second position in which the one or more elongate arms at least partially cover the proximal end of the bone anchor.

For example, in some embodiments, the first position may be a flexed position in which the one or more elongate arms are flexed outwardly from the at least one aperture of the plate. The second position may be a rest position in which the one or more elongate arms are neither flexed inwardly nor outwardly. Alternatively, in the second position, the one or more elongate arms may be flexed inwardly towards the at least one aperature of the plate for receipt within a corresponding one or more grooves formed in the top surface of the plate.

In other embodiments, the first position may be a rest position in which the one or more elongate arms are neither flexed inwardly nor outwardly. In the second position, the one or more elongate arms may be flexed inwardly for receipt within one or more grooves formed in the top surface of the plate.

In some embodiments, one or more tabs may be formed in the top surface of the bone fixation plate. Each tab may at least partially cover a top surface of one of the first and second elongate arms when the arm is seated within the bone fixation plate in its corresponding groove.

In some embodiments, the one or more elongate arms may be formed from a biocompatible metal. In some embodiments, the one or more elongate arms may be formed from a polymer.

In another aspect, a spinal fixation plate is provided with first and second apertures that overlay the same vertebral body of the spine and that permit partial passage of first and second bone anchors through the plate. Also provided are first and second elongate arms configured to flex between first and second positions. In the first position, at least one of the bone anchors can be advanced between and past the first and second elongate arms, partially through the plate via its respective aperture, and into bone. In the second position, at least one of (e.g., both) the first and second elongate arms covers at least part of a proximal end of each bone anchor advanced into bone in the first position. For example, in some embodiments, due to angulation of the bone anchor in the bone and/or the configuration of the bone fixation plate, only one of the elongate arms may cover the proximal end of the anchor.

In some embodiments, the first and second elongate arms may be formed generally in the shape of an hour-glass. In other embodiments, the first and second elongate arms may be formed generally in the shape of a FIG. 8. In some embodiments, the first and second elongate arms may be parallel. Other embodiments of wire members are also provided.

In some embodiments, the spinal fixation plate may include a part-spherical or part-conical seat adjacent to at least one of the apertures. This seat may allow for multi-angular articulation with a complimentary part-spherical or part-conical surface of a respective bone anchor.

In some embodiments, the spinal fixation plate may be configured for rigid spinal fixation. For example, the width of each aperture in the plate may be substantially equal to a width of the bone anchor that is adjacent to the aperture when the bone anchor is advanced fully into the plate.

In other embodiments, the spinal fixation plate may be configured for dynamic spinal fixation. For example, the width of each aperture may be greater than a width of the bone anchor that is adjacent to the aperture when the bone anchor is advanced fully into the plate.

In still another aspect, a spinal fixation apparatus may be provided that includes a spinal fixation plate and a wire member, where the spinal fixation plate includes a bottom surface with first and second grooves. The wire member includes first and second elongate arms that span entirely across a top surface of the plate, a third arm coupled to the first and second arms, and a fourth arm coupled to the first and second arms. The third and fourth arms are configured for receipt within the first and second grooves of the spinal fixation plate. The first and second elongate arms are flexible between a first position for permitting passage of at least one bone anchor, and a second position for resisting back out of the bone anchor from the plate. In some embodiments, the first and second elongate arms may be coupled to the third and fourth arms via a plurality of struts that extend along sides of the spinal fixation plate.

In another aspect, a method for bone fixation is provided. At least one bone anchor is advanced between and past an opposed pair of elongate arms, partially through a bone fixation plate, and into bone. The opposed pair of elongate arms is flexed inwardly to cause at least one of the elongate arms to cover at least part of a proximal end (e.g., head) of the bone anchor.

### BRIEF DESCRIPTION OF THE DRAWINGS

For a better understanding of the present invention, including the various objects and advantages thereof, reference is made to the following detailed description, taken in conjunction with the accompanying illustrative drawings, in which like reference characters refer to like parts throughout, and in which:

FIG. 1A is a perspective view of a bone fixation assembly that includes a bone fixation plate, at least one bone anchor (e.g., screw), and at least one wire member for resisting back out of the at least one bone anchor from the plate, according to some embodiments of the present invention;

FIG. 1B is a perspective view of the bone fixation assembly of FIG. 1A prior to advancement of the bone anchor past the wire member, partially through the bone fixation plate, and into bone;

FIG. 1C is a perspective view of the bone fixation assembly of FIG. 1A subsequent to advancement of the bone anchor past the wire member, partially through the bone fixation plate, and into bone;

FIG. 2A is a top view of the wire member of FIG. 1A in a rest position in which arms of the wire member are neither flexed inwardly nor outwardly;

FIG. 2B is a top view of the wire member of FIG. 1A in which the arms of the wire member are flexed outwardly relative to the rest position to allow passage of the bone anchor;

FIG. 2C is a top view of the wire member of FIG. 1A in which the arms of the wire member are flexed inwardly relative to the rest position;

FIG. 2D is a side, perspective view of the wire member of FIG. 1A;

FIG. 3A is a top view of the bone fixation plate of FIG. 1A;

FIG. 3B is a bottom view of the bone fixation plate of FIG. 1A;

FIG. 4 is a top view of another bone fixation plate according to some embodiments of the present invention; and

FIGS. 5-1C are top views of wire members and bone fixation plates according to other embodiments of the present invention.

### DETAILED DESCRIPTION OF THE INVENTION

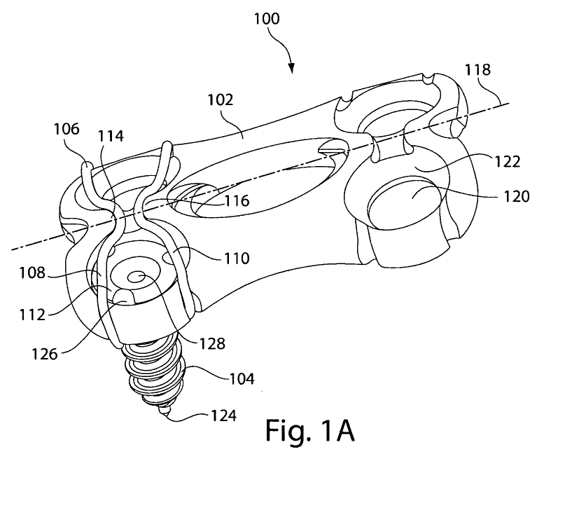

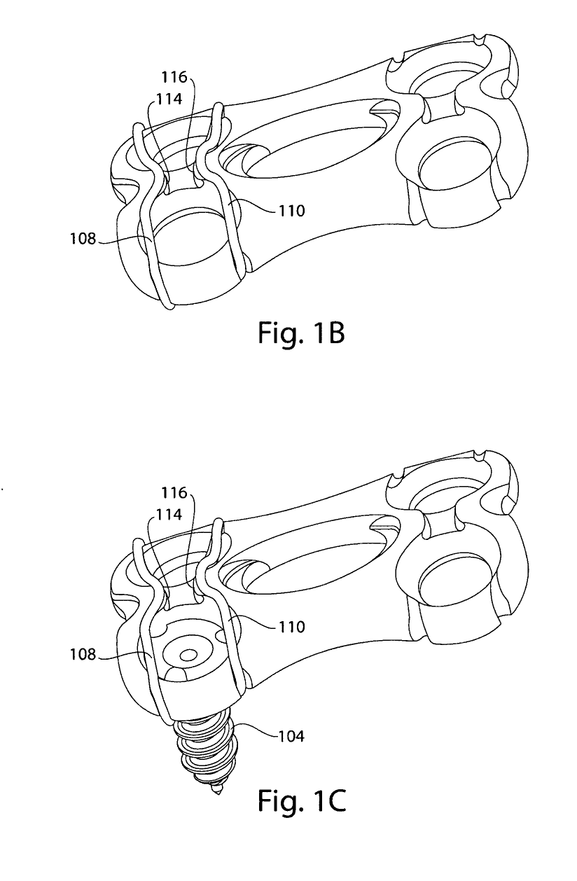

FIG. 1A is a perspective view of a bone fixation assembly 100 according to some embodiments of the present invention. Assembly 100 includes bone fixation plate 102, one or more bone anchors (e.g., screws) 104, and one or more wire members 106. Each wire member 106 may include one or more (e.g., two) elongate arms (108, 110) with each arm having, for example, a circular-, square-, or triangle-shaped cross-section. Elongate arms 108 and/or 110 may be configured to flex (e.g., elastically) between a first position in which they permit advancement of bone anchor 104 past the arms, partially through plate 102, and into bone, and a second position in which arms 108 and/or 110 resist back out of bone anchor 104 from the plate.

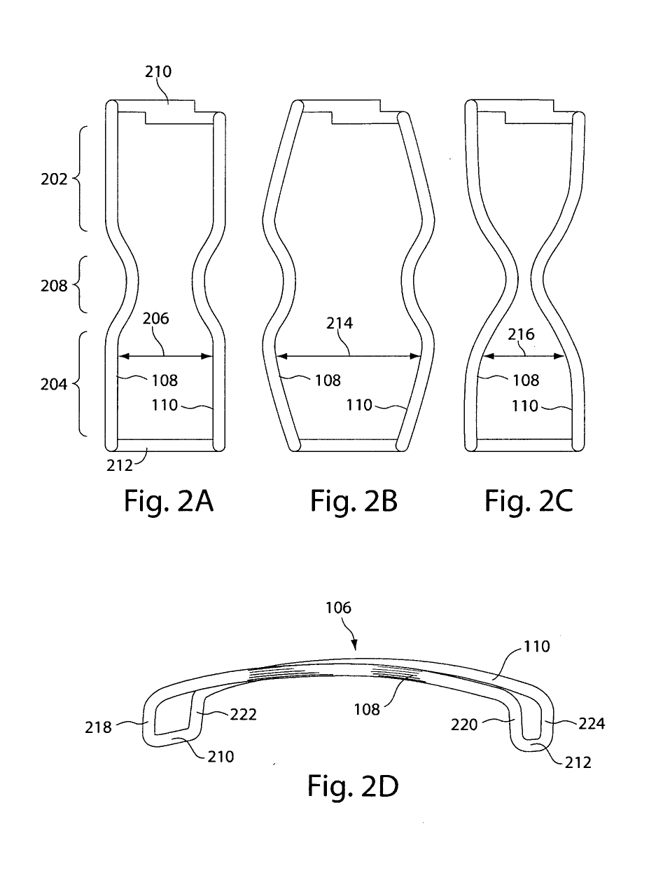

FIG. 1A shows the second position of elongate arms 108 and 110 according to some embodiments of the present invention. In the second position, one or both of elongate arms 108 and 110 may at least partially cover proximal end 112 (head) of bone anchor 104. In some embodiments, in the second position, elongate arms 108 and/or 110 may be seated within grooves 114 and/or 116, respectively, formed within a top surface of bone fixation plate 102. For example, grooves 114 and 116 may be generally opposed. In some embodiments, the cross-sectional shapes of grooves 114 and/or 116 may be complimentary to the cross-sectional shapes of elongate arms 108 and/or 110, respectively. The first position of elongate arms 108 and 110 according to some embodiments of the present invention is described in connection with FIGS. 1B, 1C, 2A, and 2B.

Bone fixation plate 102 is a one-level plate configured to span across and fixate two vertebrae of the cervical spine. Plate 102 includes two wire members 106, with each wire member spanning across two bone anchors 104, although only one wire member 106 and one bone anchor 104 are shown in FIG. 1A to avoid over-complicating the drawing. Other N-level bone fixation assemblies are also provided (e.g., N=2, 3, 4, etc.) in accordance with some embodiments of the present invention. Generally, an N-level assembly spans across and fixates N+1 vertebrae. Each N-level assembly may include, for example, N+1 wire members 106 and 2(N+1) bone anchors 104.

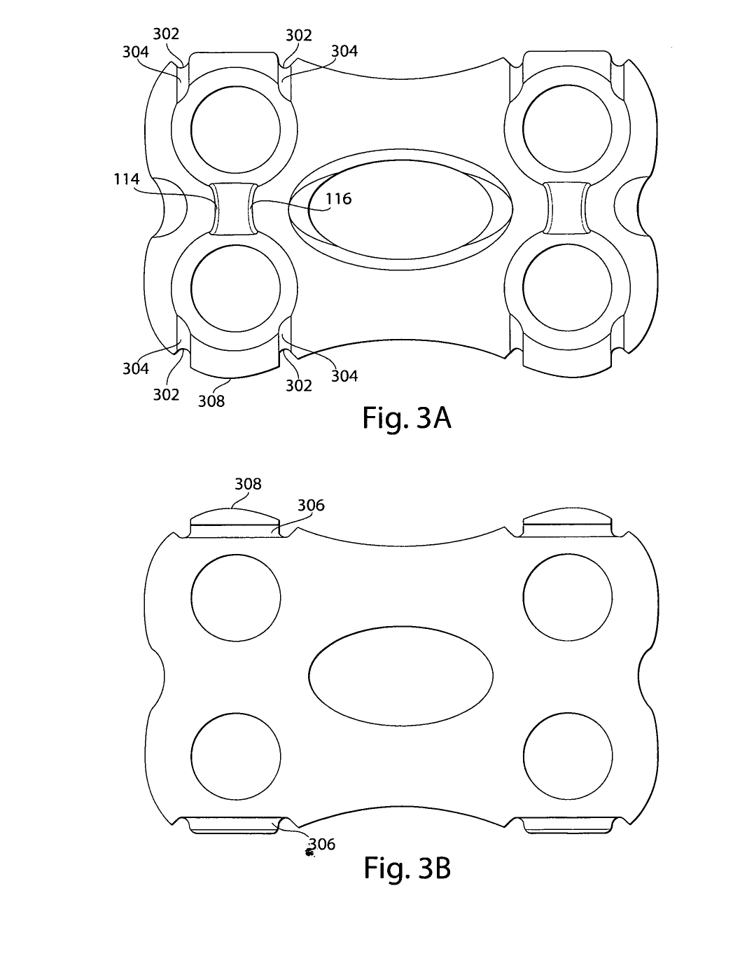

In some embodiments, each elongate arm (108, 110) of wire member 106 may span across the entire top surface of bone fixation plate 102 in a direction generally transverse to longitudinal axis 118 of the plate. When plate 102 is used for spinal fixation, longitudinal axis 118 may coincide with the axis of the spinal column. Additional components of member 106 may wrap around the sides of bone fixation plate 102 and attach to complimentary features in the bottom surface of plate 102, as is described in greater detail in connection with FIGS. 2D and 3B. Advantageously, attaching wire member 106 to plate 102 transversely to longitudinal axis 118 may allow the same size wire member 106 to be used for bone fixation plates having the same width (e.g., 18-20 millimeters (mm)), and optionally the same thickness (e.g., 1.8-2.2 mm), but different lengths (e.g., bone fixation plates of different levels (N) and/or having different lengths to account for variations in spinal anatomies). In other embodiments, wire members having different configurations may be provided for use with the same bone fixation plate or with bone fixation plates having different lengths, widths, and/or thicknesses.

Bone fixation plate 102 forms a plurality of apertures 120 that permit a corresponding plurality of bone anchors 104 to pass partially through plate 102 and into bone. For example, four apertures 120 having circular or part-circular cross-sections are provided in the one-level bone fixation plate of FIG. 1A. For other N-level bone fixation plates, 2(N+1) apertures 120 may be formed in the plate. Bone fixation plate 102 includes surface 122 located adjacent to and forming each aperture 120. Surface 122 also forms a part-spherical or part-conical seat configured for multi-angular articulation with a complimentary part-spherical or part-conical surface of bone anchor 104.

In some embodiments, the width of the inner most part of surface 122 may be approximately equal to the width of an adjacent portion of bone anchor 104 when anchor 104 is advanced fully into plate 102. This may prevent lateral movement of bone anchor 104 within plate 102 and cause rigid fixation between surface 122 and the adjacent surface of anchor 104. In other embodiments, the width of the inner most part of surface 122 may be greater than the width of the adjacent surface of bone anchor 104. This may allow for movement of bone anchor 104 within plate 102 and dynamic articulation between surface 122 and the adjacent surface of bone anchor 104. In still other embodiments, the bone fixation plate may include multiple aperture sizes that allow the same plate to be used for both rigid and dynamic fixation, at the option of the surgeon. For example, the same bone fixation plate may include a set of apertures configured for rigid fixation, and an independent set of apertures configured for dynamic fixation. Alternatively or additionally, one or more of the apertures may have, for example, an ovalized shape, and surface 122 may have an ovalized seat configured for receiving the head of the bone anchor. This may allow the bone anchor to translate as well as rotate. Only a portion of the apertures may be ovalized so that some bone anchor(s) can translate whereas others cannot.

In some embodiments, each bone anchor 104 is configured at its distal end 124 for self-tapping or self-drilling. Proximal end 112 (head) of bone anchor 104 may include a recess (e.g., having a non-circular cross-sectional shape) and/or other feature(s) for receiving a complimentary tip of a surgical tool. For example, in the embodiment of FIG. 1A, bone anchor 104 includes multiple (e.g., three equidistant) prongs 126 positioned around the perimeter of proximal end 112 and a central, circular or non-circular recess 128 for receiving a surgical tool.

Bone fixation assembly 100 and its various components may be made from any suitable material or combination of materials. For example, in some embodiments, all of components 102, 104, and 106 are made from titanium, stainless steel, and/or other biocompatible metal(s). In other embodiments, one or more (e.g., all) of components 102, 104, and 106 are made from a polymer or one or more biocompatible ceramics, such as the high strength, high toughness doped silicon nitride ceramic described in commonly-owned U.S. Pat. No. 6,881,229, which is hereby incorporated by reference herein in its entirety. In some embodiments, the one or more materials (e.g., metal or polymer) used for wire member(s) 106 may have an elastic property.

In some embodiments, bone fixation plate 102 has a lordotic curvature that corresponds to a lordotic curvature of the human cervical spine. For example, an anterior face of plate 102 may be contoured and rounded so as to reduce or eliminate irritation of the esophagus and the surrounding tissues.

In some embodiments, bone fixation plate 102 is configured to promote bone ingrowth to the plate. For example, in some embodiments, at least a portion of bone fixation plate 102 may be made from a porous material, such as the porous doped silicon nitride ceramic described in commonly-owned U.S. Pub. Appln. No. 20050049706, which is hereby incorporated by reference herein in its entirety. Alternatively or additionally, one or more bone contacting surfaces of bone fixation plate 102 may be roughened, for example, by mechanical blasting and/or plasma spraying with metal particles of one or more sizes.

In some embodiments, bone fixation plate 102 is coated with a bio-active material having an osteoconductive property, such as hydroxyapatite or a calcium phosphate material. Alternatively or additionally, bone fixation plate 102 may carry one or more therapeutic agents, for example, for enhancing bone fusion and ingrowth. Examples of such therapeutic agents include natural or synthetic therapeutic agents such as bone morphogenic proteins (BMPs), growth factors, bone marrow aspirate, stem cells, progenitor cells, antibiotics, and other osteoconductive, osteoinductive, osteogenic, bio-active, or any other fusion enhancing material or beneficial therapeutic agent. In some embodiments, bone anchor(s) 104 and/or wire member(s) 106 may be porous, roughened, and/or coated with one or more bio-active and/or therapeutic materials.

FIG. 1B is a perspective view of bone fixation assembly 100 prior to advancement of bone anchor 104 past (e.g., between) arms 108 and 110 of wire member 106 and into bone fixation plate 102. As shown, bone fixation assembly 100 is in an unlocked state in which elongate arms 108 and 110 of member 106 are not seated within the opposed grooves 114 and 116 of plate 102. Rather, arms 108 and 110 are positioned on the top surface of bone fixation plate 102 outwardly of grooves 114 and 116. In some embodiments, the separation of elongate arms 108 and 110 in the unlocked state may be less than the diameter of proximal end 112 (head) of bone anchor 104. Thus, with further reference to FIGS. 2A and 2B, wire member 106 may flex outwardly from a rest position (FIGS. 1B and 2A), in which arms 108 and 110 are neither flexed inwardly nor outwardly, to a flexed position (FIG. 2B) in which proximal end 112 (head) of bone anchor 104 can pass through member 106. In other embodiments, the separation of arms 108 and 110 in the rest position may be greater than or equal to the diameter of proximal end 112 of bone anchor 104. In such embodiments, bone anchor 104 may pass between arms 108 and 110 of wire member 106 without the need for outward flexing of the arms.

FIG. 1C is a perspective view of bone fixation assembly 100 subsequent to insertion of bone anchor 104 past arms 108 and 110 of wire member 106 and into bone fixation plate 102, but prior to locking of bone anchor 104 within the assembly. Due to an elastic property of wire member 106, member 106 may return to same rest position shown in FIGS. 1B and 2A automatically subsequent to bone anchor 104 passing between arms 108 and 110 of member 106.

FIG. 1A shows the locked state of bone fixation assembly 100 in which arms 108 and 110 are seated within grooves 114 and 116 of bone fixation plate 102. Locking of bone anchor 104 within assembly 100 may be achieved by flexing arms 108 and 110 of wire member 106 inwardly from the rest position (FIGS. 1C and 2A) to place arms 108 and 110 within grooves 114 and 116 of plate 102 (FIGS. 1A and 2C).

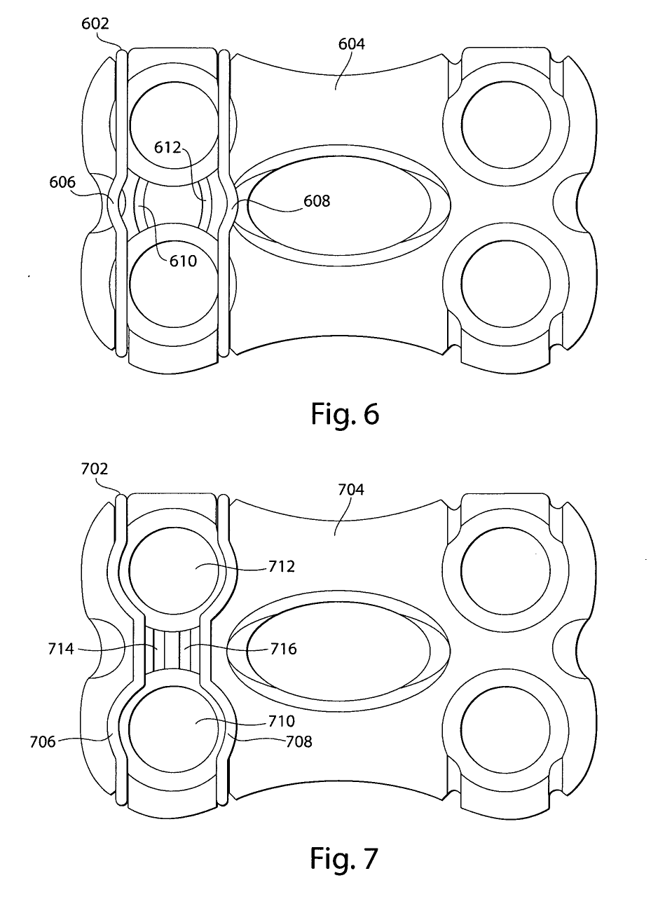

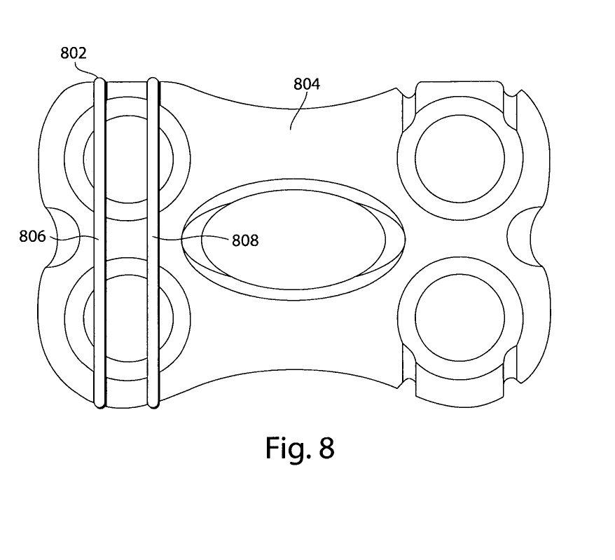

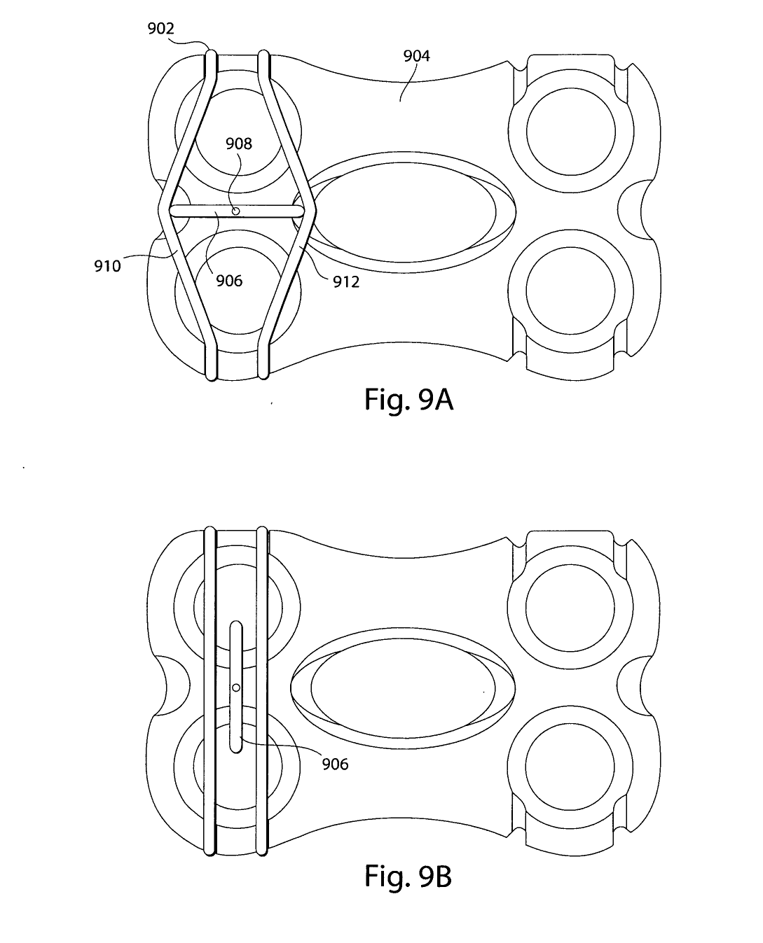

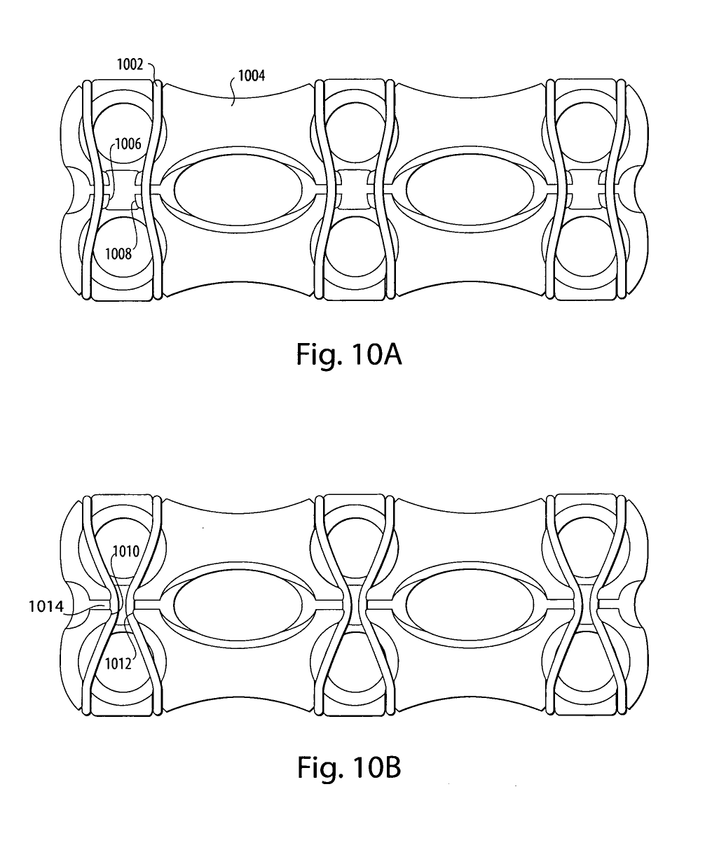

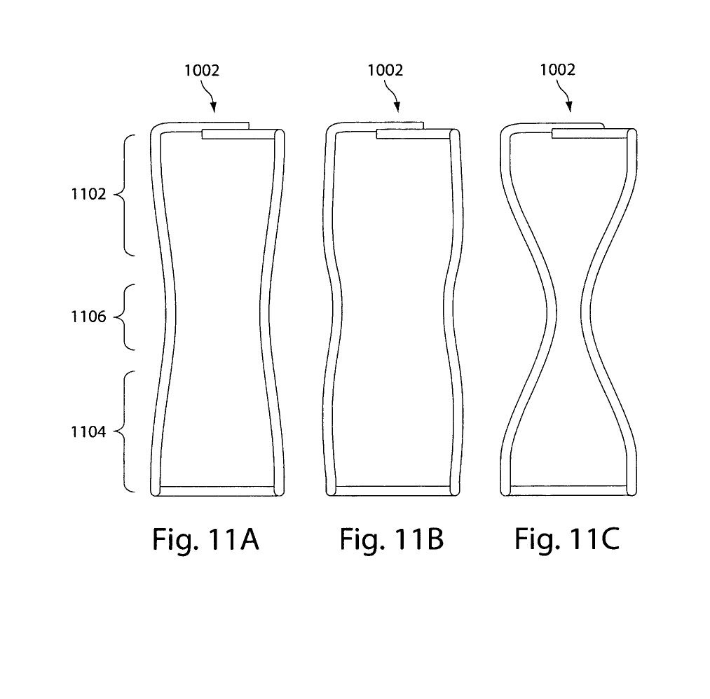

FIG. 2A is a top view of wire member 106 in the rest position in which arms 108 and 110 are neither flexed inwardly nor outwardly. In this embodiment, wire member 106 is formed generally in the shape of an hour-glass. Other configurations of wire members according to some embodiments of the present invention are described in connection with FIGS. 5-9B. Wire member 106 includes first region 202 and second region 204 configured for placement immediately adjacent to first and second apertures 120 of bone fixation plate 102. In the rest position of wire member 106, the portions of arms 108 and 110 in regions 202 and 204 are separated by distance 206 and are generally parallel. Wire member 106 also includes third region 208 between regions 202 and 204. In some embodiments, third region 208 may include curved portions of arms 108 and 110 configured for receipt within corresponding curved grooves 114 and 116 of bone fixation plate 102. In this embodiment, the curved portions of arms 108 and 110 in region 206 have the same shape (e.g., part-circular shape), but opposite concavity.

Wire member 106 also may include arm 210 in region 202 and arm 212 in region 204. Arms 210 and 212 may be coupled to and positioned generally transversely to arms 108 and 110. In some embodiments, arms 210 and 212 may be configured for attachment to complimentary grooves in a bottom surface of plate 102 (FIG. 3B).

FIG. 2B is a top view of wire member 106 in which arms 108 and 110 are flexed outwardly relative to the rest position of FIG. 2A to allow bone anchor 104 to pass between the arms. Distance 214 between arms 108 and 110 in regions 202 and 204 of wire member 106, which may be greater than or equal to the diameter of proximal end 112 (head) of bone anchor 104, may be increased relative to distance 206 between the arms in the rest position. The outward flexing of arms 108 and 110 may result from interaction of arms 108 and 110 with a surface (e.g., part-spherical or part-conical bottom surface) of bone anchor 104 as anchor 104 is advanced between arms 108 and 110, partially through plate 102, and into bone. Alternatively or additionally, a surgical tool (e.g., screw guide or sheath) may be provided that causes arms 108 and 110 to flex outwardly.

FIG. 2C is a top view of wire member 106 in a locked position in which the curved portions of arms 108 and 110 are flexed inwardly relative to the rest position of FIG. 2A to cause the arms to be seated within grooves 114 and 116 of bone fixation plate 102. Distance 216 between arms 108 and 110 in regions 202 and 204 of wire member 106, which may be less than the diameter of proximal end 112 (head) of bone anchor 104, may be reduced relative to distance 206 between the arms in the rest position. In some embodiments, a surgical tool (e.g., clamp) may be provided that causes arms 108 and 110 to flex inwardly from the rest position to the locked position. For example, such a tool may grip and apply an inwardly directed force to the curved portions of member 106 in region 208.

In some embodiments, due to an elastic property of wire member 106, member 106 may return fully or partially to the rest position (FIGS. 1C and 2A) from the locked position (FIGS. 1 A and 2C) in response to removal of arms 108 and 110 from grooves 114 and 116. The arms may be removed from grooves 114 and 116, for example, by gripping and applying an inwardly (e.g., and simultaneously upwardly) directed force to the portions of member 106 in region 208. In other approaches, such a force may be applied to regions 202 and/or 204. This force may cause arms 108 and 110 to clear grooves 114 and 116 and then flex elastically back to the rest position. The surgeon may have access to the bone anchor(s) 104 immediately, for example, when distance 206 between arms 108 and 110 in the rest position is greater than the diameter of proximal end 112 of bone anchor 104. Otherwise, the surgeon may have access to the bone anchors subsequent to an application of force that causes arms 108 and 110 to flex outwardly (FIG. 2b). In another approach, a surgeon may access bone anchor(s) 104 by cutting arms 108 and/or 110.

FIG. 2D is a perspective view of wire member 106. As shown, wire member 106 may includes struts 218, 220, 222, and 224 for coupling arms 108 and 110 of member 106 to arms 210 and 212. Struts 218 and 220 may couple first and second ends of arm 108 to arm 210 and arm 212, respectively. Struts 222 and 224 my couple first and second ends of arm 110 to arm 210 and arm 212, respectively. Struts 218-224 may wrap around the sides of bone fixation plate 102. In some embodiments (e.g., FIG. 3A), the sides of plate 102 may include grooves for receiving struts 218-224.

In some embodiments, wire member 106 may be formed by bending or otherwise forming a single elongate rod (e.g., cylindrical rod having a diamater from about 0.5 mm to about 0.75 mm) into the configuration shown in FIGS. 2A-2D. Arm 210 of member 106 may be formed by bonding (e.g., welding) together first and second ends of the rod, which may cause arm 210 to have twice the thickness of arm 212. Alternatively, the ends of the rod may be beveled to reduce the thickness of arm 210, for example, to equal to or slightly greater than the thickness of arm 212. In other embodiments, multiple rods or other components may be bonded together to form wire member 106. In still other embodiments, wire member 106 may be formed (e.g., molded or cut) as a single, continuous component.

FIG. 3A is a top view of bone fixation plate 102. As shown, curved grooves 114 and 116 for receiving complimentary curved portions of wire member 106 may be formed within the top surface of bone fixation plate 102. Alternatively or additionally, grooves 302 for receiving struts 218-224 of wire member 106 (FIG. 2D) may be formed within side surfaces of bone fixation plate 102. The top surface of plate 102 may also form a recessed channel 304 adjacent to each groove 302. In some embodiments, the cross-sectional shapes of grooves 302 may be complimentary to the cross-sectional shapes of struts 218-224. The cross-sectional shapes of recessed channels 304 may be complimentary to the cross-sectional shapes of arms 108 and 110.

FIG. 3B is a bottom view of bone fixation plate 102. As shown, grooves 306 for receiving arms 210 and 212 of wire member 106 (FIG. 2A) may be formed within the bottom surface of bone fixation plate 102. In some embodiments, the cross-sectional shapes of grooves 306 may be complimentary to the cross-sectional shapes of arms 210 and 212. This may decrease stress on the arms by providing a smooth contact surface. This may also increase the frictional forces between bone fixation plate 102 and arms 210 and 212 that resist ejection of arms 210 and 212 from grooves 304. Still further, since arms 108 and 110 are held in tension across the top surface of the bone fixation plate, this may also increase the force needed to eject arms 108 and 110 from grooves 114 and 116. In some embodiments, plate 102 also may include one or more tabs 308 adjacent to one or more of grooves 306. Subsequent to seating arm 210 and/or arm 212 within a corresponding groove 306, a tab 308 adjacent to that groove may be deformed in the direction of the arm. This may crimp arms 210 and/or 212 in place and further resist their ejection from grooves 306. In some embodiments, when only one of arms 210 and 212 (e.g., 210) is to be crimped in place, the groove 306 for that arm may be formed (e.g., cut) deeper in the bottom surface of bone fixation plate 102 than the groove 306 for the other arm. In some embodiments, wire member 106 may be attached to bone fixation plate as part of the manufacturing process. In other embodiments, wire member 106 and plate 102 may be provided separately for assembly, for example, by the surgeon.

In some embodiments, the thickness of the assembly of bone fixation plate 102 and wire member 106 may be less than the sum of their individual thicknesses. For example, in one embodiment, the total thickness resulting from the assembly of a bone fixation plate 102 having a thickness of 2.15 mm and a wire member 106 (e.g., cylindrical wire member) having a diameter of 0.75 mm is 2.6 mm. This reduction in total thickness may result from the inclusion of grooves 114 and 116 (FIG. 3A), recessed channels 304 (FIG. 3A), and/or grooves 306 (FIG. 3B) within surfaces of plate 102. For example, in one embodiment, each of recessed channels 304 may have a depth of about 0.30 mm. Alternatively or additionally, grooves 114, 116, and/or 306 may have a depth greater than or equal to about 0.75 mm. Advantageously, the total thickness of the assembly of wire member 106 and bone fixation plate 102 according to some embodiments of the present invention may be less than, for example, the thickness of a bone fixation plate that forms internal cavit(ies) for housing members that resist back out of bone anchors.

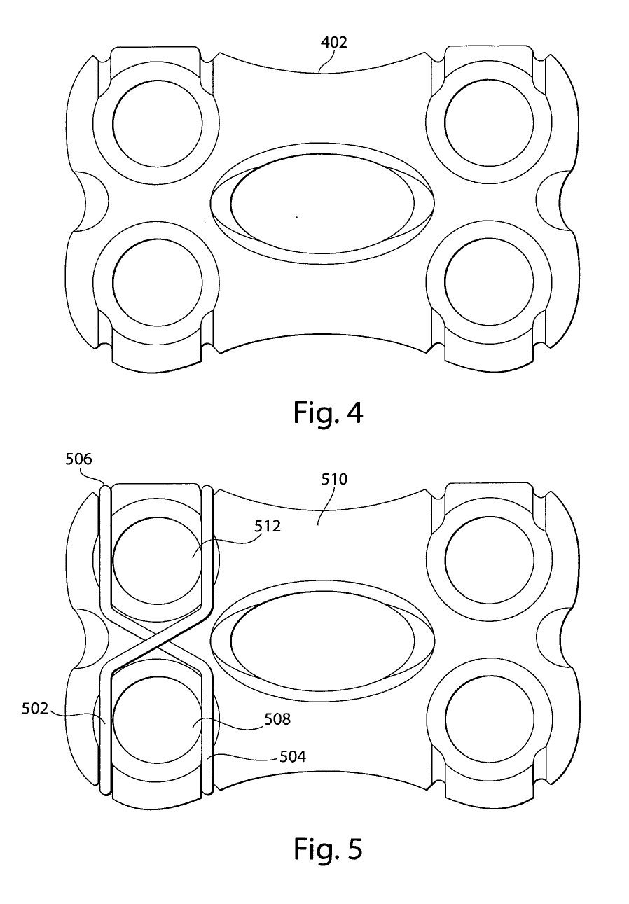

FIG. 4 shows another embodiment of a bone fixation plate 402 in accordance with the present invention. Bone fixation plate 402 may be the same or similar to plate 102 (FIG. 3A) in all respects, except that bone fixation plate 402 may lack grooves 114 and 116 for locking arms of a wire member into place relative to plate 402. For example, bone fixation plate 402 may be used in connection with wire member 106 or another wire member (e.g., FIGS. 5-9B) that flexes from a rest position (e.g., FIG. 2A) to a flexed position (e.g., FIG. 2B) to allow passage of a bone anchor, and then returns to the rest position in which the member may at least partially cover and resist back out of the bone anchor from plate 402. Other variations of bone fixation plate 102 are, of course, possible. For example, in some embodiments, a bone fixation plate may be provided that includes at least one feature (e.g., tab) in the top surface of the plate for preventing an arm of a wire member (e.g., arm 108) from flexing, while allowing another arm of the wire member (e.g., arm 110) to flex freely. For example, the free arm may flex from a rest position to at least one other position, such as an outwardly flexed position for permitting passage of a bone anchor and/or a locked position for resisting back out of the anchor.

FIGS. 5-9B are top views of wire members and bone fixation plates according to other embodiments of the present invention. In FIG. 5, arms 502 and 504 of wire member 506 are configured generally in the shape of a figure-8. For example, flexing arms 502 and 504 outwardly in the region of aperture 508 of bone fixation plate 510 may cause arms 502 and 504 to flex inwardly in the region of aperture 512, and vice versa. Bone fixation plate 510 may be the same or similar to bone fixation plate 402 (FIG. 4). Wire member 506 may be configured to flex (e.g., elastically) from a rest position (FIG. 5), to an outwardly flexed position to allow passage of a bone anchor, and back to the rest position in which member 506 resists back out of the bone anchor from plate 510. In other embodiments, bone fixation plate 510 may include one or more grooves formed within the top surface of the plate for locking arms 502 and/or 504 into place. In some embodiments, wire member 506 and plate 510 may be modified to place arms 502 and 504 of member 506 closer together (i.e., more overlap of apertures 508 and/or 512) in the rest position.

FIG. 6 is a top view of a wire member 602 and a bone fixation plate 604 according to another embodiment of the present invention. Wire member 602 and plate 604 may be the same or similar to wire member 106 and bone fixation plate 102 (FIG. 1A) in all respects, except that the curves in portions 606 and 608 of member 602 and grooves 610 and 612 in plate 604 may be opposite to (i.e., reverse concavity of) the corresponding portions and grooves in member 106 and plate 102.

FIG. 7 is a top view of a wire member 702 and a bone fixation plate 704 according to yet another embodiment of the present invention. Again, wire member 702 and plate 704 may be the same or similar to wire member 106 and bone fixation plate 102 (FIG. 1A) in all respects, except that arms 706 and 708 of wire member 702 may be curved (e.g., part-circular) in the regions of apertures 710 and 712. An additional difference is that the center portion of wire member 702, and corresponding grooves 714 and 716, may be straight and generally parallel.

FIG. 8 is a top view of a wire member 802 and a bone fixation plate 804 according to another embodiment of the present invention. Wire member 802 may be the same or similar to wire member 106 (FIG. 1A) in all respects, and plate 804 may be the same or similar to plate 402 (FIG. 4), except that arms 806 and 808 of wire member 802 may be straight and closer together in the rest position. Wire member 802 may be configured to flex (e.g., elastically) from a rest position (FIG. 8), to an outwardly flexed position to allow passage of a bone anchor, and back to the rest position in which member 802 resists back out of the bone anchor from plate 804. In other embodiments, bone fixation plate 804 may include one or more grooves (e.g., similar to grooves 714 and 716 (FIG. 7)) for locking arms 806 and/or 808 into place. In still other embodiments, wire member 802 or another wire member (e.g., FIGS. 2A, 5, 6, and/or 7) may be rigid and may be attached to the bone fixation plate by a surgeon subsequent to advancement of the bone anchor(s) into the plate.

FIGS. 9A and 9B are top views of a wire member 902 and a bone fixation plate 904 according to yet another embodiment of the present invention. Wire member 902 may be the same or similar to wire member 802 (FIG. 8) in all respects. Bone fixation plate 904 may be the same or similar to plate 804 in all respects, except that plate 904 may additionally include lever 906 attached to the plate, for example, by pin 908. Lever 906 may be configured to rotate from a first position (FIG. 9A) in which the lever causes arms 910 and 912 to flex outwardly to permit passage of a bone anchor, to a second position (FIG. 9B) in which arms 910 and 912 at least partially cover and resist back out of the bone anchor. In some embodiments, in the second position of lever 906, the lever also may at least partially cover and resist back out of the bone anchor.

FIGS. 10A and 10B are top views of a wire member 1002 and a bone fixation plate 1004 according to another embodiment of the present invention. Wire member 1002 may be the same or similar to wire member 106 (FIG. 1A) in all respects, except that wire member 1002 may have a different hour-glass shape. Additional details are described in connection with FIGS. 11A-C below. Bone fixation plate 1004 may be the same or similar to bone fixation plate 102 (FIG. 1A) in all respects, except that bone fixation plate may be a two-level plate. Additionally, one or both of grooves 1006 and 1008 formed within the top surface of plate 1004 may have adjacent tabs 1010 and 1012 that at least partially overlay top surfaces of the arms of wire member 1002 when member 1002 is seated within the grooves. For example, tabs 1010 and 1012 (e.g., about 0.25 mm long) may cover about one half the top surface of the arms of member 1002. In some embodiments, grooves 1006 and 1008 and corresponding tabs 1010 and 1012 may be separated (e.g., intersected) by a recessed surface 1014, such as a channel having a flat bottom surface. Surface 1014 may allow for the passage of a tool that grips and applies an inwardly and/or upwardly or downwardly directed force to the curved portions of member 1002 in its center region, to either insert the arms of member 1002 into grooves 1006 and 1008 or remove them from the grooves. In FIG. 10A, wire member 1002 is in a rest position in which its arms are not flexed inwardly or outwardly. In FIG. 10B, the arms of wire member 1002 are flexed inwardly and seated within grooves 1006 and 1008.

FIGS. 1A-C are top views of wire member 1002. FIG. 11A shows wire member 1002 in a rest position in which its arms are neither flexed inwardly nor outwardly. In regions 1102 and 1104 of wire member 1002, the arms are curved slightly inwardly toward the center region 1106. In region 1106, the arms of wire member 1002 have less curvature than the curvature of wire member 106 in corresponding region 208 (FIG. 2A). FIG. 1B shows shows wire member 1002 in which its arms are flexed slightly outwardly relative to the rest position of FIG. 11A to allow a bone anchor to pass between the arms. FIG. 11C shows wire member 1002 in a locked position in which the arms are flexed inwardly to cause the arms to be seated within the grooves of bone fixation plate 1004.

Thus it is seen that bone fixation plates wire members are provided for resisting back out of associated bone anchors. Although particular embodiments have been disclosed herein in detail, this has been done by way of example for purposes of illustration only, and is not intended to be limiting with respect to the scope of the appended claims, which follow. In particular, it is contemplated that various substitutions, alterations, and modifications may be made without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. The applicant reserves the right to pursue such inventions in later claims.

What is claimed is:

1. A bone fixation apparatus, comprising:
a bone fixation plate comprising a top surface, a bottom surface, and at least one aperture between the top surface and the bottom surface for permitting partial passage of at least one bone anchor through the plate, the bone fixation plate further comprising a length in one direction and a width in another direction, wherein the width is less than the length; and
a member for retaining the at least one bone anchor within the at least one aperture of the plate, the member comprising one or more elongate arms configured to span across at least a portion of the top surface of the plate in a direction substantially transverse to the direction of the length of the plate, wherein the one or more elongate arms are configured to flex between:
a first position in which the at least one bone anchor can be advanced past the one or more elongate arms, partially through the plate, and into bone; and
a second position in which the one or more elongate arms at least partially cover a proximal end of the at least one bone anchor.

2. The bone fixation apparatus of claim 1, wherein the one or more elongate arms are configured to flex at least partially elastically between the first position and the second position.

3. The bone fixation apparatus of claim 1, wherein the first position is a flexed position in which the one or more elongate arms are flexed outwardly from the at least one aperture of the plate.

4. The bone fixation apparatus of claim 3, wherein the bone fixation plate further comprises one or more grooves formed in the top surface of the plate, and wherein the one or more elongate arms are received within the one or more grooves in the second position but not in the first position.

5. The bone fixation apparatus of claim 4, wherein the second position is a flexed position in which the one or more elongate arms are flexed inwardly towards the at least one aperture of the plate.

6. The bone fixation apparatus of claim 3, wherein the second position is a rest position in which the one or more elongate arms are neither flexed inwardly nor outwardly.

7. The bone fixation apparatus of claim 1, wherein the first position is a rest position in which the one or more elongate arms are neither flexed inwardly nor outwardly.

8. The bone fixation apparatus of claim 7, wherein the bone fixation plate further comprises one or more grooves formed in the top surface of the plate, and wherein the one or more elongate arms are received within the one or more grooves in the second position but not in the first position.

9. The bone fixation apparatus of claim 1, wherein the one or more elongate arms span across the entire width of the bone fixation plate.

10. The bone fixation apparatus of claim 1, wherein the one or more elongate arms comprise metal.

11. The bone fixation apparatus of claim 1, further comprising the at least one bone anchor, wherein the at least one bone anchor comprises at least one bone screw.

12. A spinal fixation apparatus, comprising:
a spinal fixation plate comprising a top surface, a bottom surface, and first and second apertures between the top surface and the bottom surface for permitting partial passage of respective first and second bone anchors through the plate, wherein the first and second apertures are configured to overlay the same vertebral body of the spine; and
first and second elongate arms configured to flex between:
a first position in which at least one of the bone anchors can be advanced between and past the first and second elongate arms, partially through the plate via the respective at least one of the apertures, and into bone; and
a second position in which at least one of the first and second elongate arms covers at least part of a proximal end of each bone anchor advanced into bone in the first position.

13. The spinal fixation apparatus of claim 12, wherein the first and second elongate arms are formed generally in the shape of an hour-glass.

14. The spinal fixation apparatus of claim 12, wherein at least one of the apertures has a circular cross-section.

15. The spinal fixation apparatus of claim 12, wherein the first and second elongate arms are configured to flex at least partially elastically between the first position and the second position.

16. The spinal fixation apparatus of claim 12, wherein the spinal fixation plate further comprises first and second grooves formed in the top surface of the plate, and wherein the first and second elongate arms are received within the first and second grooves, respectively, in the second position but not in the first position.

17. The spinal fixation apparatus of claim 16, wherein the top surface of the spinal fixation plate adjacent to at least one of the first and second grooves at least partially covers a top surface of a corresponding one of the first and second elongate arms when the arm is seated within its corresponding groove.

18. The spinal fixation apparatus of claim 12, wherein the spinal fixation plate comprises a part-spherical or part-conical seat adjacent to at least one of the apertures for receiving a complimentary surface of the respective bone anchor.

19. The spinal fixation apparatus of claim 12, further comprising at least one of the bone anchors, wherein a width of the respective aperture is substantially equal to a width of a portion of the bone anchor that is adjacent to the aperture when the bone anchor is advanced fully into the plate.

20. The spinal fixation apparatus of claim 12, further comprising at least one of the bone anchors, wherein a width of the respective aperture is greater than a width of a portion of the bone anchor that is adjacent to the aperture when the bone anchor is advanced fully into the plate.

21. The spinal fixation apparatus of claim 12, wherein the first elongate arm is coupled to the second elongate arm.

22. A spinal fixation apparatus, comprising:
a spinal fixation plate comprising a top surface, a bottom surface comprising first and second grooves, and first and second apertures between the top surface and the bottom surface for permitting partial passage of respective first and second bone anchors through the plate; and
a member for retaining the bone anchors within the plate, the member comprising first and second elongate arms spanning entirely across the top surface of the plate, a third arm coupled to the first and second elongate arms, and a fourth arm coupled to the first and second elongate arms, wherein the third arm and the fourth arm are configured for receipt within the first and second grooves of the spinal fixation plate, and wherein the first and second elongate arms are flexible between:
a first position in which at least one of the bone anchors can be advanced between and past the first and second elongate arms, partially through the plate via the respective at least one of the apertures, and into bone; and
a second position in which at least one of the first and second elongate arms covers at least part of a proximal end of each bone anchor advanced into bone in the first position.

23. The spinal fixation apparatus of claim 22, wherein the first and second elongate arms are coupled to the third arm and to the fourth arm via a plurality of struts extending along sides of the spinal fixation plate.

24. A method for bone fixation, comprising:
advancing at least one bone anchor between and past an opposed pair of elongate arms, partially through a bone fixation plate, and into bone; and
flexing the opposed pair of elongate arms inwardly relative to one another to cause at least one of the elongate arms to cover at least part of a proximal end of the bone anchor.

25. A bone fixation apparatus, comprising:
means extending in a direction substantially transverse to a longitudinal axis of a bone fixation plate for at least partially covering a top surface of the plate, comprising means for flexing between (i) a first position in which at least one bone anchor can be advanced partially through the plate via at least one aperture and into bone, and (ii) a second position in which the means resists back out of the bone anchor from the plate.
